(19) United States
(12) Patent Application Publication
(10) Pub. No.: US 2010/0104646 A1
(43) Pub. Date: Apr. 29, 2010

(54) NANOPARTICLE COMPOSITION FOR PREVENTION OF HAIR LOSS AND PROMOTION OF HAIR GROWTH

(76) Inventor: Ji Hwal Kim, Seoul (KR)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 12/520,951

(22) PCT Filed: Dec. 24, 2007
(86) PCT No.: PCT/KR07/06781

§ 371 Date: Dec. 10, 2009

(30) Foreign Application Priority Data
Dec. 28, 2006 (KR) 10-2006-0136223

(51) Int. Cl. A61K 9/14; A61K 36/258; A61P 43/00

(52) U.S. Cl. 424/489; 424/728

(57) ABSTRACT

A nanoparticle composition is provided for preventing hair loss and promoting hair growth. It is based on herbal materials and thus has no harmful side effects on the body. When applied to the scalp, the nanoparticle composition deeply infiltrates into the dermis to promote blood circulation therein and provide nutrients thereto. Accordingly, the nanoparticle composition stimulates and activates hair follicles to thus promote the metabolism of hairs. Also, the nanoparticle composition exerts antioxidant activity on the scalp to thus inhibit depilation and aid in hair regrowth.

### TECHNICAL FIELD

The present invention relates, in general, to a nanoparticle composition for the prevention of hair loss and the promotion of hair growth and, more particularly, to a nanoparticle composition which promotes the metabolism of hairs and exerts antioxidant activity on the scalp, thus preventing depilation and promoting hair regrowth.

### BACKGROUND ART

In a human body are approximately 100,000˜150,000 hair follicles. Each hair grows in cycles of various phases consisting of anagen, catagen and telogen.

For scalp hair, the cycle is completed in around 3 to 6 years, with an average hair loss of about 60-100 strands a day.

Generally, alopecia refers to abnormal hair loss scientifically described as an increase in the number of hair follicles in the catagen or telogen phase compared to the anagen phase.

Causes of alopecia are known to include abnormality of the endocrine system, such as hormonal imbalance, abnormality of the autonomous nervous system, a decrease in blood flow in hair follicles, hypersecretion of the male hormone testosterone, hypersecretion of sebum, functional decrease of the scalp by bacteria such as Pityrosporum sp., malnutrition of hair follicles, allergy, genetic factors, aging, stress, etc.

However, the definite mechanism of alopecia has remained unclear thus far. The proportion of the population that suffers from hair loss has been increasing due to changes in dietary life and increased social and environmental stress. Recently, alopecia has spread to youth in their twenties and thirties as the scalp and the hair are injured by the overuse of shampoo, hair gel, perm, hair dye, hair dryers, etc. An increasing population of women is also reported to suffer from hair loss.

The treatment of alopecia is attracting worldwide attention. In this regard, studies are focused on chemical therapy with female hormones, blood circulation-promoting agents, vitamin E, vitamin B2 complex, carotene, germicides, etc. and hair transplantation.

For example, clinical studies showed that Propecia, the brand name for a drug developed by Merck, prevented hair loss in 68% of the participants taking the drug, and 66% of them enjoyed hair regrowth. However, Propecia is effective only as long as it is taken; the hair gained or maintained is lost within several months of ceasing therapy. In addition, this medicine is very expensive and has side effects of loss of sexual function and impotence.

Minoxidil is a vasodilator and was originally used as an oral drug to treat high blood pressure. It was, however, discovered to have the interesting side-effect of promoting hair growth and reversing baldness, and Pharmacia & Upjohn produced a topical solution that contained 2% minoxidil to be used to treat baldness and hair loss, under the brand name Rogaine. Treatments usually include 5% concentration solutions, as the patent rights for Minoxidil have expired.

As a drug to combat hair loss, the most common side effect is an itchy scalp. In addition, other side effects of minoxidil include irritation of the application area, rash, dry skin, flushing of the skin due to widening of the small blood vessels (erythema), allergic contact dermatitis, unwanted growth of hair on other parts of the body, very low blood pressure, irregular or fast heart beat, etc.

Conventional compositions, as described above, are not sufficiently effective in preventing hair loss and promoting hair regrowth. In fact, the commercially available medicines may cause increased hair loss due to the side effects of inflammation or soreness at the roots of the hair.

### DISCLOSURE

### Technical Problem

Accordingly, the present invention has been made keeping in mind the above problems occurring in the prior art, and an object of the present invention is to provide a nanoparticle composition for the prevention of hair loss and the promotion of hair growth, comprised essentially of natural herbal extracts with no side effects on the body, which, when applied to the scalp, deeply infiltrates into the dermis to promote blood circulation therein and provide nutrients thereto. Accordingly, the nanoparticle composition stimulates and activates hair follicles to promote the metabolism of hairs. Also, the nanoparticle composition of the present invention exerts antioxidant activity on the scalp to inhibit depilation and promote hair regrowth.

### Technical Solution

In order to accomplish the above object, the present invention provides a nanoparticle composition for the prevention of hair loss and the promotion of hair growth, comprising lecithin-capsuled nanoparticles prepared from an admixture consisting of a first mixture based on herbal extracts and a second mixture based on vitamins.

### ADVANTAGEOUS EFFECTS

The nanoparticle composition of the present invention is based on herbal materials and thus has no harmful side effects on the body. When applied to the scalp, the nanoparticle composition of the present invention deeply infiltrates into the dermis to promote blood circulation therein and provide nutrients thereto. Accordingly, the nanoparticle composition stimulates and activates hair follicles to promote the metabolism of hairs. Also, the nanoparticle composition of the present invention exerts antioxidant activity on the scalp to inhibit depilation and promote hair regrowth. Therefore, the nanoparticle composition of the present invention finds various applications in the cosmetics industry, including functional shampoos, hair sprays, etc.

### BRIEF DESCRIPTION OF DRAWINGS

FIG. 1 shows the structure of a nanoparticle useful in the composition of the present invention in schematic views.

FIG. 2 shows the infiltration of the nanoparticle composition of the present invention into the dermis of the scalp in a schematic view.

FIG. 3 shows nanoparticles of the present invention in a photograph.

### BEST MODE

In accordance with an aspect thereof, the present invention provides a nanoparticle composition for the prevention of hair loss and the promotion of hair growth, comprising lecithin-capsuled nanoparticles prepared from an admixture consisting of a first mixture and a second mixture. The first mixture comprises 9˜10% by weight of a Salvia miltiorrhiza extract, 9˜10% by weight of a safflower extract, 10˜12% by weight of a Zanthoxylum piperitum extract, 10˜12% by weight of a Torilis japonica fruit extract, 12˜14% by weight of a Cnidium officinale Makino extract, 5.5˜6% by weight of a green tea leaf extract, 5.5˜6% by weight of a pomegranate extract, 7˜8% by weight of pine tree leaf extract, 1.5˜2% by weight of a red ginseng extract, 1.5˜2% by weight of a ginseng extract, and 17˜18% by weight of an Angelicae extract. The second mixture comprises 91% by weight of distilled water and 9% by weight of a vitamin mixture of nicotinamide and tocopherol acetate. The admixture is prepared by mixing the first mixture and the second mixture at a weight ratio of 4:6.

Then, the admixture is passed through a 100-mesh filter to remove impurities and large particles. The filtrate is again passed through a 0.8 μm filter and subsequently through a 0.45 μm filter to afford nanoparticles.

These nanoparticles are mixed with soybean lecithin at a ratio of 1:1, followed by treatment under a pressure of 1000 bar in a high-pressure homogenizer to produce lecithin-capsuled nanoparticles.

### [Mode for Invention]

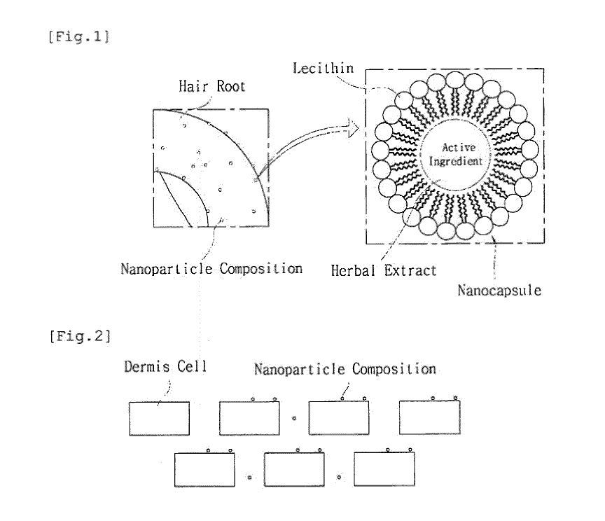

A detailed description will be given of preferred embodiments of the present invention with reference to FIGS. 1 to 3.

In accordance with the present invention, a nanoparticle composition based on herbal materials with no materials that are poisonous to the body is provided for preventing hair loss and promoting hair regrowth. When applied to the scalp, the nanoparticle composition of the present invention deeply infiltrates into the dermis to promote blood circulation therein and provide nutrients thereto. Accordingly, the nanoparticle composition stimulates and activates hair follicles to promote the metabolism of hairs. Also, the nanoparticle composition of the present invention exerts antioxidant activity on the scalp to inhibit depilation and aid hair regrowth.

### Example 1

A first mixture was made by combining 9˜10% by weight of a Salvia miltiorrhiza extract, 9˜10% by weight of a safflower extract, 10˜12% by weight of a Zanthoxylum piperitum extract, 10˜12% by weight of a Torilis japonica fruit extract, 12˜14% by weight of a Cnidium officinale Makino extract, 5.5˜6% by weight of a green tea leaf extract, 5.5˜6% by weight of a pomegranate extract, 7˜8% by weight of pine tree leaf extract, 1.5˜2% by weight of a red ginseng extract, 1.5˜2% by weight of a ginseng extract, and 17˜18% by weight of an Angelicae extract. Separately, a second mixture was prepared from 91% by weight of distilled water and 9% by weight of a vitamin mixture of nicotinamide and tocopherol acetate. The first and the second mixture were combined with each other at a weight ratio of 4:6 to produce an admixture.

Then, the admixture was filtered through a 100-mesh filter to remove impurities and large particles. The filtrate thus obtained was again passed through a 0.8 μm filter and subsequently through a 0.45 μm filter to afford nano-size particles.

Afterwards, these nanoparticles was mixed with soybean lecithin at a ratio of 1:1, followed by treatment under a pressure of 1000 bar in a high-pressure homogenizer to produce lecithin-capsuled nanoparticles.

The constituents of the first mixture, including the Salvia miltiorrhiza extract, the safflower extract, the Zanthoxylum piperitum extract, the Torilis japonica fruit extract, the Cnidium officinale Makino extract, the green tea leaf extract, the pomegranate extract, the pine tree leaf extract, the red ginseng extract, the ginseng extract, and the Angelicae extract, were prepared by decocting the respective materials in water.

Filtration through a 100 mesh filter, a 0.8 μm filter and a 0.45 μm filter in sequence removed impurities and large particles from the admixture, leaving extracts containing nanoparticles.

The nanoparticle extract was encapsulated with lecithin. A mixture of 1:1 the nanoparticle extract:lecithin was treated under a pressure of 1,000 bar in a high-pressure homogenizer to produce lecithin capsules with the herb extracts contained therein.

Lecithin, which may be extracted from soy beans, has a structure similar to that of intercellular lipids. Therefore, when the lecithin-capsuled nanoparticles are applied to the scalp, lecithin helps the extract particles deeply infiltrate into the dermis or cuticle layer of the scalp to improve the effect of the herbal extract.

The properties and features of the materials constituting the first mixture are as follows.

*Salvia miltiorrhiza: grows in shady places and originates from China. Two or three, egg-like small nuts per plant. The roots smell peculiar, taste bitter and are useful in herbal medicine. They are applied to the treatment of irregular menstruation, menstrual pains, afterpain, blood-static abdominal pain, bruises, insomnia and rashes.

*Cnidium officinale Makino: cultured as a medicinal herb and originates from China. In herbal medicine, the roots are prescribed for headaches, anemia, and women's diseases thanks to the medicinal effects of tranquilization and pain relief. It is aromatic enough to protect clothes from moths. Also, it shows excellent resuscitative effects on cells, as expressed by the saying that dying pine trees are restored to life again when a hot-water extract of Cnidium officinale Makino is applied to the roots.

*Zanthoxylum piperitum: a deciduous shrub belonging to the Zanthoxylum family, which grows to about 3 m with many thorny sprigs. In Korea, young stems and leaves are used, together with unripe fruits, as food materials. Extracts from fruits are widely used as starter fluid and seasoning. In addition to industrial applications, it is used as a honey source and a medicinal material. In folk remedies, powders of its fruits, bark or leaves are kneaded along with flour and vinegar into dough, which is then applied as a patch with paper or cloth to the treatment of mastitis, tumors and bruises. Hemorrhoids are treated by washing with a decoction of roots and root-burn ashes. A decoction of Zanthoxylum piperitum is also used in the treatment of headaches and coughs and as a painkiller for dental caries.

In herbal medicine, it is prescribed for arm and leg pain and soreness and numbness associated with cold. Having stimulatory germicidal activity and a warm characteristic, Zanthoxylum piperitum is described as having medicinal activities of strengthening the stomach, preventing paralysis, and promoting urination. Administration of a pasty decoction thereof at a dose of three spoonfuls a day is prescribed for edema.

*Safflower: long used as a natural dye and known to treat bone fractures. While metal platinum (Pt) may be a fatal poison to humans and animals, the organic platinum in safflower seeds has been found to have surprising medicinal activity against cancers, bone fractures, osteoporosis, etc.

Safflower oil is composed predominantly of linoleic acid and glyceride, with a high content of anti-aging vitamin E. Safflower is of warm nature and acts to nourish the blood, so that it is used for the treatment of static blood and menstrual pain. Its flowers are known to promote the production and destruction of blood cells. The application of a safflower extract to the scalp promotes the blood circulation of the scalp.

*Angelicae: grown wild or artificially cultivated. The stems and leaves grown in the early spring are cooked as salads and the roots are dried for use as a medicine. Chubby roots are more medicinally effective. Showing haematogenesis, Angelicae is typically prescribed for nervous diseases, blood diseases, and low vitality. It is also used for the treatment of heartburn, vaginal hernias and infertility. Other examples of target diseases or symptoms of Angelicae include various inflammations, bruises from injuries with ironware and diarrheic pain and body temperature fluctuation. In addition, it invigorates the internal organs and makes the flesh lustystimulates the libido??. While the root head is useful in the treatment of bruises, rootlets and root hairs control hemorrhaging. Having potent haematogenic activity, Angelicae is the most widely used herbal medicine. When applied to the scalp, Angelicae is expected to promote blood circulation in the scalp.

*Pomegranate: originating from West Asia and the Northwest regions of India. About 40% constituted of carbohydrates (glucose, fructose) with citric acid at a content of about 1.5%. It also contains water-soluble vitamins (B1, B2, niacin acid), but in low amounts. Tannin is found in the shell. Being rich in estrogen, the seeds are prescribed for menopausal disorders.

Both the shell and the seeds are useful in the prevention of hypertension and arteriosclerosis and in the treatment of women's diseases and swelling, and particularly in the treatment of diarrhea. Its volatile alkaloid is applied to the preparation of medicines against parasite worms, especially tapeworms. The fruit juice has a pretty color useful for the preparation of wine, beverages and confectionaries. When mixed with olive oil, it is effective for the relief of constipation.

Green tea leaf: epicatechin-3-gallate and epigallocatechin-3-gallate, contained in green tea leaves, selectively inhibit 5-alpha reductase. Rich in catechins, which are reported to show anti-androgen activity, they are thus preventive of hair loss. Depilation or hair decolorization occurs when lipid superoxides, produced through the oxidation of unsaturated fatty acids of foods, destroy cell membranes. Together with vitamin C, catechins are antioxidant enough to inhibit the activity of lipid superoxides and thus to suspend hairs from aging. Having alkaline minerals and vitamins B and C, green tea leaves suppress the production of melanin and can make the skin elastic and smooth.

When applied to the scalp, a green tea leaf extract endows hair roots with antioxidant activity.

*Ginseng: rich in saponin glycosides such as ginsenosides, steroids, vitamin B complex, choline, etc. In herbal medicine, ginseng is generally used for medicinally effective activities including a tonic, a cardiotonic, a peptic, an immunostimulant, a hematic and the like.

The application of a ginseng extract to the scalp activates the cells.

*Torilis japonica: The shoots are used as a salad ingredient. The fruits are used as astringents, antiphlogistics, and pesticides.

*Red ginseng: red ginseng is found to be sedative and stimulative to the central nervous system. In the circulatory system, it is preventive of hypertension and arteriosclerosis. Also, the medicinal effects of red ginseng include haematogenic activity, a decrease in blood sugar level, hepatoprotective activity, stimulation of the endocrine system to arouse sexual desire, anti-inflammation, anti-tumor activity, radio-protection, and skin-soothing activity.

One of the most important effects of red ginseng comes from the adaptogen that it contains. The adaptogen is a natural herb product that is scientifically verified to increase the body's resistance to stresses such as trauma, anxiety and bodily fatigue.

Accordingly, a red ginseng extract, when applied to the scalp, affords the hair root cells protection against stress and promotes smooth blood circulation thanks to the haematogenesis activity.

*Pine tree leaf: It is described in a Korean ancient herb medicine book that the long-term ingestion of uncooked pine tree leaves provides various effects including anti-aging, invigoration, resistance to hair decolorization, and cold resistance. Another Korean traditional pharmaceutical book also describes that when ingesting a powder of pine tree leaves, persons are invigorated and can bear the cold.

Among the ingredients of pine tree leaves, there are terpenes that are volatile. More than 40 terpenes have been found in pine tree leaves. Most prevalent are alpha-pinene, beta-pinene, beta-phellandrene, beta-caryophyllene, myrcene, camphene, and alpha-terpinolene. Broadleaf trees contain respective characteristic terpenes, but needle-shaped trees are much more abundant in terpenes. According to herbal folklore, pine tree leaves taste bitter and are warm in temperature with no poisons, and the medicinal effects are focused on the heart and the spleen. Recent pharmaceutical experiments conducted in China and North Korea show that extracts from pine tree leaves promote intracellular redox processes, prevent stringent inflammation and control hemorrhaging.

When applied to the scalp, a pine tree leaf extract is expected to activate the cellular metabolism thanks to its ability to promote intracellular redox processes.

In contrast to the herbal materials of the first mixture, including Salvia miltiorrhiza, safflower, Zanthoxylum piperitum, Torilis japonica fruit, Cnidium officinale Makino, green tea leaf, pomegranate, pine tree leaf, red ginseng, ginseng, and Angelicae, the vitamins of the second mixture are well known for their properties and features, and thus a description thereof is omitted.

### Example 2

A dry material comprising 6 parts by weight of Salvia miltiorrhiza, 1 part by weight of ginseng, 7 parts by weight of Zanthoxylum piperitum, 6 parts by weight of safflower, 12 parts by weight of Angelicae, 8 parts by weight of Cnidium officinale Makino, 4 parts by weight of a pomegranate shell, 4 parts by weight of green tea leaves, 7 parts by weight of a Torilis japonica fruit, 1 part by weight of red ginseng, and 3 parts by weight of pine tree leaves was mixed with water at a ratio of 1:10, and the mixture was boiled at 80˜90° C. for 3˜4 hrs. The supernatant was allowed to stand at room temperature.

The extract thus obtained was mixed with 6 parts by weight of a vitamin mixture and then passed through a 100-mesh filter to remove impurities and large particles therefrom. The remaining filtrate was filtered through a 0.8 μm filter and a 0.45 μm filter sequentially to afford a nanoparticle extract.

Afterwards, the nanoparticle extract was mixed with soybean lecithin at a ratio of 1:1, followed by treatment under a pressure of 1000 bar in a high-pressure homogenizer to produce lecithin-capsuled nanoparticles.

With reference to FIG. 1, there is a schematic view showing the nanoparticle composition of the present invention infiltrated into a hair root, with an enlarged view showing a herbal nanoparticle extract encapsulated in lecithin.

With reference to FIG. 2, there is a schematic view showing that the nanoparticle composition of the present invention is deeply infiltrated into the dermis when it is applied to the scalp.

When applied to the scalp, the nanoparticle composition of the present invention can deeply infiltrate into the dermis because of the small size thereof, to thus promote blood circulation therein and provide nutrients thereto. Accordingly, the nanoparticle composition stimulates and activates hair follicles to promote the metabolism of hairs. Also, the nanoparticle composition of the present invention exerts antioxidant activity on the scalp to inhibit depilation and aid hair regrowth.

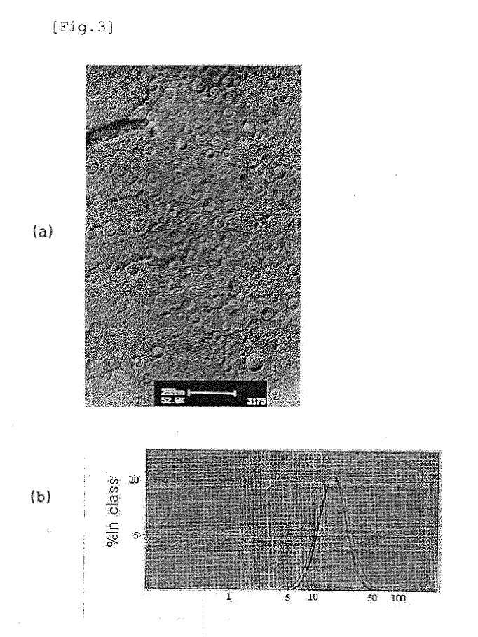

Referring to FIG. 3A, there is an electron photograph of the nanoparticle composition after freeze-drying. FIG. 3B shows the particle size distribution of the nanoparticle composition according to the present invention.

As shown in FIG. 3A, the nanoparticle composition of the present invention is in the form of nanocapsules. As is apparent from the graph, the nanoparticles are distributed within a size range from 5 to 50 nm.

### INDUSTRIAL APPLICABILITY

As described hitherto, the nanoparticle composition of the present invention is based on herbal materials and thus has no harmful side effects on the body. When applied to the scalp, the nanoparticle composition of the present invention deeply infiltrates into the dermis to promote blood circulation therein and provide nutrients thereto. Accordingly, the nanoparticle composition stimulates and activates hair follicles to thus promote the metabolism of hairs. Also, the nanoparticle composition of the present invention exerts antioxidant activity on the scalp to thus inhibit depilation and aid in hair regrowth. Therefore, the nanoparticle composition of the present invention finds various applications in the cosmetic industry, including in functional shampoos, hair sprays, etc.

1. A nanoparticle composition for prevention of hair loss and promotion of hair growth, prepared by:
Combining a first mixture with a second mixture at a weight ratio of 4:6 to give an admixture, said first mixture comprising 9˜10% by weight of a Salvia miltiorrhiza extract, 9˜10% by weight of a safflower extract, 10˜12% by weight of a Zanthoxylum piperitum extract, 10˜12% by weight of a Torilis japonica fruit extract, 12˜14% by weight of a Cnidium officinale Makino extract, 5.5˜6% by weight of a green tea leaf extract, 5.5˜6% by weight of a pomegranate extract, 7˜8% by weight of pine tree leaf extract, 1.5˜2% by weight of a red ginseng extract, 1.5˜2% by weight of a ginseng extract, and 17˜18% by weight of an Angelicae extract, said second mixture comprising 91% by weight of distilled water and 9% by weight of a vitamin mixture of nicotinamide and tocopherol acetate,
passing the admixture through a 100-mesh filter to remove impurities and large particles and filtering a filtrate remaining through a 0.8 μm filter and subsequently through a 0.45 μm filter to afford nanoparticles, and
mixing the nanoparticles with soybean lecithin at a ratio of 1:1 to give an intermediate mixture and treating the intermediate mixture under a pressure of 1000 bar in a high-pressure homogenizer to produce lecithin-capsuled nanoparticles.

2. A nanoparticle composition for prevention of hair loss and promotion of hair growth, prepared by:
mixing a dry material comprising 6 parts by weight of Salvia miltiorrhiza, 1 part by weight of ginseng, 7 parts by weight of Zanthoxylum piperitum, 6 parts by weight of safflower, 12 parts by weight of Angelicae, 8 parts by weight of Cnidium officinale Makino, 4 parts by weight of a pomegranate shell, 4 parts by weight of green tea leaves, 7 parts by weight of a Torilis japonica fruit, 1 part by weight of red ginseng, and 3 parts by weight of pine tree leaves with water at a ratio of 1:10, boiling the water containing the dry material at 80˜90° C. for 3˜4 hrs to extract ingredients from the materials, cooling the extract at room temperature, mixing the extract with 6 parts by weight of vitamins, passing a mixture of the extract and the vitamins through a 100-mesh filter to remove impurities and large particles therefrom and give a filtrate, and filtering the filtrate through a 0.8 μm filter and a 0.45 μm filter sequentially to afford a nanoparticle extract, and
mixing the nanoparticle extract with soybean lecithin at a ratio of 1:1 and then treating the nanoparticle-lecithin mixture under a pressure of 1000 bar in a high-pressure homogenizer to produce lecithin-capsuled nanoparticles.
